(19) United States
(12) Patent Application Publication
(10) Pub. No.: US 2015/0297579 A1
(43) Pub. Date: Oct. 22, 2015

(54) TREATMENT OF CANCER WITH POMALIDOMIDE IN A RENALLY IMPAIRED SUBJECT

(71) Applicant: CELGENE CORPORATION, Summit, NJ (US)

(72) Inventor: Claudia Eve KASSERRA, Morris Plains, NJ (US)

(21) Appl. No.: 14/440,333

(22) PCT Filed: Nov. 4, 2013
(86) PCT No.: PCT/US13/68237

§ 371 Date: May 1, 2015

Related U.S. Application Data
(60) Provisional application No. 61/764,466, filed on Feb. 13, 2013.
(60) Provisional application No. 61/722,722, filed on Nov. 5, 2012.

(51) Int. Cl. A61K 31/454; A61K 31/573

(52) U.S. Cl. CPC A61K 31/454; A61K 31/573

(57) ABSTRACT

Provided herein are methods of treating, preventing, or managing one or more symptoms of a disease (e.g., cancer) in a subject with renal impairment, comprising administering to the subject pomalidomide. Also provided herein are methods of treating, preventing, or managing one or more symptoms of a disease (e.g., cancer) in a subject with renal impairment, comprising administering to the subject a therapeutically effective amount of pomalidomide and dexamethasone.

### 1. CROSS REFERENCE TO RELATED APPLICATIONS

This application claims priority to U.S. Provisional Application Nos. 61/722,722, filed Nov. 5, 2012; and 61/764,466, filed Feb. 13, 2013; the disclosure of each of which is incorporated herein by reference in its entirety.

### 2. FIELD

Provided herein are methods of treating, preventing, or managing one or more symptoms of a disease (e.g., cancer) in a renally impaired subject, comprising administering to the subject pomalidomide. Also provided herein are methods of treating, preventing, or managing one or more symptoms of a disease (e.g., cancer) in a renally impaired subject, comprising administering to the subject a therapeutically effective amount of pomalidomide and dexamethasone.

### 3. BACKGROUND

### 3.1 Pathobiology of Cancer

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Roitt et al., Immunology, 17.1-17.12 (3rd ed., Mosby, St. Louis, Mo., 1993).

There is an enormous variety of cancers which are described in detail in the medical literature. Examples include cancer of the lung, colon, rectum, prostate, breast, brain, and intestine. The incidence of cancer continues to climb as the general population ages, as new cancers develop, and as susceptible populations (e.g., people infected with AIDS or excessively exposed to sunlight) grow. A tremendous demand therefore exists for new methods and compositions that can be used to treat patients with cancer.

Many types of cancers are associated with new blood vessel formation, a process known as angiogenesis. Several of the mechanisms involved in tumor-induced angiogenesis have been elucidated. The most direct of these mechanisms is the secretion by the tumor cells of cytokines with angiogenic properties. Examples of these cytokines include acidic and basic fibroblastic growth factor (a,b-FGF), angiogenin, vascular endothelial growth factor (VEGF), and TNF-α. Alternatively, tumor cells can release angiogenic peptides through the production of proteases and the subsequent breakdown of the extracellular matrix where some cytokines are stored (e.g., b-FGF). Angiogenesis can also be induced indirectly through the recruitment of inflammatory cells (particularly macrophages) and their subsequent release of angiogenic cytokines (e.g., TNF-α, b-FGF).

Lymphoma refers to cancers that originate in the lymphatic system. Lymphoma is characterized by malignant neoplasms of lymphocytes—B lymphocytes and T lymphocytes (i.e., B-cells and T-cells). Lymphoma generally starts in lymph nodes or collections of lymphatic tissue in organs, including, but not limited to, the stomach or intestines. Lymphoma may involve the marrow and the blood in some cases. Lymphoma may spread from one site to other parts of the body.

The treatment of various forms of lymphomas are described, for example, in U.S. Pat. No. 7,468,363, the disclosure of which is incorporated herein by reference in its entirety. Such lymphomas include, but are not limited to, Hodgkin's lymphoma, non-Hodgkin's lymphoma, cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, and low grade follicular lymphoma.

Non-Hodgkin's lymphoma (NHL) is the fifth most common cancer for both men and women in the United States, with an estimated 63,190 new cases and 18,660 deaths in 2007. Jemal et al., CA Cancer J. Clin. 2007; 57(1):43-66. The probability of developing NHL increases with age and the incidence of NHL in the elderly has been steadily increasing in the past decade, causing concern with the aging trend of the US population. Id. Clarke et al., Cancer 2002; 94(7):2015-2023.

Diffuse large B-cell lymphoma (DLBCL) accounts for approximately one-third of non-Hodgkin's lymphomas. While some DLBCL patients are cured with traditional chemotherapy, the remainder die from the disease. Anticancer drugs cause rapid and persistent depletion of lymphocytes, possibly by direct apoptosis induction in mature T and B cells. Stahnke et al., Blood 2001; 98:3066-3073. Absolute lymphocyte count (ALC) has been shown to be a prognostic factor in follicular non-Hodgkin's lymphoma and recent results have suggested that ALC at diagnosis is an important prognostic factor in diffuse large B-cell lymphoma. Kim et al., Journal of Clinical Oncology, 2007; 25:18S.

Leukemia refers to malignant neoplasms of the blood-forming tissues. Various forms of leukemias are described, for example, in U.S. Pat. No. 7,393,862, the disclosure of which is incorporated herein by reference in its entirety. Although viruses reportedly cause several forms of leukemia in animals, causes of leukemia in humans are to a large extent unknown. The Merck Manual, 944-952 (17^{th} ed. 1999). Transformation to malignancy typically occurs in a single cell through two or more steps with subsequent proliferation and clonal expansion. In some leukemias, specific chromosomal translocations have been identified with consistent leukemic cell morphology and special clinical features (e.g., translocations of 9 and 22 in chronic myelocytic leukemia, and of 15 and 17 in acute promyelocytic leukemia). Acute leukemias are predominantly undifferentiated cell populations and chronic leukemias more mature cell forms.

Acute leukemias are divided into lymphoblastic (ALL) and non-lymphoblastic (ANLL) types. The Merck Manual, 946-949 (17^{th} ed. 1999). They may be further subdivided by their morphologic and cytochemical appearance according to the French-American-British (FAB) classification or according to their type and degree of differentiation. The use of specific B- and T-cell and myeloid-antigen monoclonal antibodies are most helpful for classification. ALL is predominantly a childhood disease which is established by laboratory findings and bone marrow examination. ANLL, also known as acute myelogenous leukemia or acute myeloblastic leukemia (AML), occurs at all ages and is the more common acute leukemia among adults; it is the form usually associated with irradiation as a causative agent.

Chronic leukemias are described as being lymphocytic (CLL) or myelocytic (CML). The Merck Manual, 949-952 (17^{th} ed. 1999). CLL is characterized by the appearance of mature lymphocytes in blood, bone marrow, and lymphoid organs. The hallmark of CLL is sustained, absolute lymphocytosis (>5,000/μL) and an increase of lymphocytes in the bone marrow. Most CLL patients also have clonal expansion of lymphocytes with B-cell characteristics. CLL is a disease of middle or old age. In CML, the characteristic feature is the predominance of granulocytic cells of all stages of differentiation in blood, bone marrow, liver, spleen, and other organs. In the symptomatic patient at diagnosis, the total white blood cell (WBC) count is usually about 200,000/μL, but may reach 1,000,000/μL. CML is relatively easy to diagnose because of the presence of the Philadelphia chromosome.

In addition to the acute and chronic categorization, neoplasms are also categorized based upon the cells giving rise to such a disorder into precursor or peripheral. See e.g., U.S. Pat. App. Publ. No. 2008/0051379, the disclosure of which is incorporated herein by reference in its entirety. Precursor neoplasms include ALLs and lymphoblastic lymphomas and occur in lymphocytes before they have differentiated into either a T- or B-cell. Peripheral neoplasms are those that occur in lymphocytes that have differentiated into either T- or B-cells. Such peripheral neoplasms include, but are not limited to, B-cell CLL, B-cell prolymphocytic leukemia, lymphoplasmacytic lymphoma, mantle cell lymphoma, follicular lymphoma, extranodal marginal zone B-cell lymphoma of mucosa-associated lymphoid tissue, nodal marginal zone lymphoma, splenic marginal zone lymphoma, hairy cell leukemia, plasmacytoma, diffuse large B-cell lymphoma, and Burkitt lymphoma. In over 95 percent of CLL cases, the clonal expansion is of a B cell lineage. See Cancer: Principles & Practice of Oncology (3rd Edition) (1989) (pp. 1843-1847). In less than 5 percent of CLL cases, the tumor cells have a T-cell phenotype. Notwithstanding these classifications, however, the pathological impairment of normal hematopoiesis is the hallmark of all leukemias.

Multiple myeloma (MM) is a cancer of plasma cells in the bone marrow. Normally, plasma cells produce antibodies and play a key role in immune function. However, uncontrolled growth of these cells leads to bone pain and fractures, anemia, infections, and other complications. Multiple myeloma is the second most common hematological malignancy, although the exact causes of multiple myeloma remain unknown. Multiple myeloma causes high levels of proteins in the blood, urine, and organs, including, but not limited to, monoclonal protein (M-protein) and other immunoglobulins (antibodies), albumin, and beta-2-microglobulin. M-protein also known as paraprotein is a particularly abnormal protein produced by the myeloma plasma cells and can be found in the blood or urine of almost all patients with multiple myeloma. Patients who have exhausted lenalidomide and bortezomib treatment have a poor prognosis. A significant proportion of patients has renal impairment with increasing incidence during disease course.

Skeletal symptoms, including bone pain, are among the most clinically significant symptoms of multiple myeloma. Malignant plasma cells release osteoclast stimulating factors (including IL-1, IL-6, and TNF), which cause calcium to be leached from bones causing lytic lesions; hypercalcemia is another symptom. The osteoclast stimulating factors, also referred to as cytokines, may prevent apoptosis, or death of myeloma cells. Fifty percent of patients have radiologically detectable myeloma-related skeletal lesions at diagnosis. Other common clinical symptoms for multiple myeloma include polyneuropathy, anemia, hyperviscosity, infections, and renal insufficiency. Renal impairment is a common comorbidity for patients with multiple myeloma, occurring in over 40% of multiple myeloma patients. Eleftherakis-Papapiakovou et al., Leuk Lymphoma 2011, 52:2299-2303.

Solid tumors are abnormal masses of tissue that may, but usually do not contain cysts or liquid areas. Solid tumors may be benign (not cancer) or malignant (cancer). Different types of solid tumors are named for the type of cells that form them. Examples of types solid tumors include, but are not limited to, malignant melanoma, adrenal carcinoma, breast carcinoma, renal cell cancer, carcinoma of the pancreas, non-small-cell lung carcinoma (NSCLC), and carcinoma of unknown primary.

The link between cancer an altered cellular metabolism has been well established. Cairns et al., Nature Rev. 2011; 11:85-95. Understanding tumor cell metabolism and the associated genetic changes thereof may lead to the identification of improved methods of cancer treatment. Id. For example, tumor cell survival and proliferation via increased glucose metabolism has been linked to the PI3K pathway, whereby mutations in tumor suppressor genes such as PTEN activate tumor cell metabolism. Id. AKT1 (a.k.a., PKB) stimulates glucose metabolism associated with tumor cell growth by various interactions with PFKFB3, ENTPD5, mTOR and TSC2 (a.k.a., tuberin). Id.

Transcription factors HIF1 and HIF2 are largely responsible for cellular response to low oxygen conditions often associated with tumors. Id. Once activated, HIF1 promotes tumor cell capacity to carry out glycolysis. Id. Thus, inhibition of HIF1 may slow or reverse tumor cell metabolism. Activation of HIF1 has been linked to PI3K, tumor suppressor proteins such as VHL, succinate dehydrogenase (SDH) and fumarate hydratase. Id. The oncogenic transcription factor MYC has also been linked to tumor cell metabolism, specifically glycolysis. Id. MYC also promotes cell proliferation by glutamine metabolic pathways. Id.

AMP-activated protein kinase (AMPK) functions as a metabolic check point which tumor cells must overcome in order to proliferate. Id. Several mutations have been identified which suppress AMPK signaling in tumor cells. Shackelford et al., Nature Rev. Cancer 2009; 9: 563-575. STK11 has been identified as a tumor suppressor gene related to the role of AMPK. Cairns et al., Nature Rev. 2011; 11:85-95.

The transcription factor p53, a tumor suppressor, also has an important role in the regulation of cellular metabolism. Id. The loss of p53 in tumor cells may be a significant contributor to changes in tumor cell metabolism to the glycolytic pathway. Id. The OCT1 transcription factor, another potential target for chemotherapeutics, may cooperate with p53 in regulating tumor cell metabolism. Id.

Pyruvate kinase M2 (PKM2) promotes changes in cellular metabolism which confer metabolic advantages to cancer cells by supporting cell proliferation. Id. For example, lung cancer cells which express PKM2 over PKM1 have been found to have such an advantage. Id. In the clinic, PKM2 has been identified as being overexpressed in a number of cancer types. Id. Thus, PKM2 may be a useful biomarker for the early detection of tumors.

Mutations in isocitrate dehydrogenases IDH1 and IDH2 have been linked to tumorigenesis, specifically, in glioblastoma and acute myeloid leukemia. Mardis et al., N. Engl. J. Med. 2009; 361: 1058-1066; Parsons et al., Science 2008; 321: 1807-1812.

### 3.2 Methods of Treating Cancer

Current cancer therapy may involve surgery, chemotherapy, hormonal therapy, and/or radiation treatment to eradicate neoplastic cells in a patient. Stockdale, 1998, Medicine, vol. 3, Rubenstein and Federman, eds., Chapter 12, Section IV. Recently, cancer therapy could also involve biological therapy or immunotherapy. All of these approaches may pose significant drawbacks for a patient. Surgery, for example, may be contraindicated due to the health of a patient or may be unacceptable to a patient. Additionally, surgery may not completely remove neoplastic tissue. Radiation therapy is only effective when the neoplastic tissue exhibits a higher sensitivity to radiation than normal tissue. Radiation therapy can also often elicit serious side effects. Hormonal therapy is rarely given as a single agent. Although hormonal therapy can be effective, it is often used to prevent or delay recurrence of cancer after other treatments have removed the majority of cancer cells. Certain biological and other therapies are limited in number and may produce side effects such as rashes or swellings, flu-like symptoms, including fever, chills and fatigue, digestive tract problems, or allergic reactions.

With respect to chemotherapy, there are a variety of chemotherapeutic agents available for treatment of cancer. A number of cancer chemotherapeutics act by inhibiting DNA synthesis, either directly or indirectly by inhibiting the biosynthesis of deoxyribonucleotide triphosphate precursors, to prevent DNA replication and concomitant cell division. Gilman et al., Goodman and Gilman's: The Pharmacological Basis of Therapeutics, Tenth Ed. (McGraw Hill, New York).

Despite availability of a variety of chemotherapeutic agents, chemotherapy has many drawbacks. Stockdale, Medicine, vol. 3, Rubenstein and Federman, eds., ch. 12, sect. 10, 1998. Almost all chemotherapeutic agents are toxic, and chemotherapy causes significant and often dangerous side effects, including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanisms from those of the drugs used in the specific treatment. This phenomenon is referred to as multidrug resistance. Because of the drug resistance, many cancers prove refractory to standard chemotherapeutic treatment protocols.

The kidney is the major excretory organ for many antineoplastic drugs and their metabolites. See, e.g., de Jonger et al., Semin. Oncol. 2006, 33, 68-73; Stevens et al., N. Engl. J. Med. 2006, 354, 2473-2483. Thus, impaired renal function, by decreasing the rate of drug elimination, may unintendedly prolong the duration of the exposure to antineoplastic drugs and may subsequently enhance the toxicity of those antineoplastic drugs. Id. Several factors can potentiate renal dysfunction and contribute to the nephrotoxicity of antineoplastic drugs, including age older than 60 years, hypertension, diabetes, cardiovascular disease, and family history of renal diseases. Id. Patients with multiple myeloma are also at risk for prerenal uremia from hyperviscosity syndrome. Id.

The effects of renal impairment on the pharmacokinetics and pharmacodynamics of drugs is complex. Renal failure not only alters the renal elimination but also the nonrenal disposition of drugs that are extensively metabolized by the liver. See, e.g., Sun et al., Pharmacol. Ther. 2006, 109, 1-11. Renal failure also impairs the liver uptake of drugs. Id.

Therefore, there exists a need for safe and effective methods of treating and managing cancer, particularly for cancer that is refractory to standard treatments, such as surgery, radiation therapy, chemotherapy and hormonal therapy, while reducing or avoiding the toxicities and/or side effects associated with the conventional therapies.

### 4. SUMMARY

Provided herein is a method of treating, preventing, or managing one or more symptoms of a disease in a subject with renal impairment, comprising administering to the subject pomalidomide.

In one embodiment, provided herein is a method of treating, preventing, or managing one or more symptoms of cancer in a subject with renal impairment, comprising administering to the subject pomalidomide.

In another embodiment, provided herein is a method of treating, preventing, or managing one or more symptoms of hematological cancer in a subject with renal impairment, comprising administering to the subject pomalidomide.

In yet another embodiment, provided herein is a method of treating, preventing, or managing one or more symptoms of multiple myeloma in a subject with renal impairment, comprising administering to the subject pomalidomide.

In yet another embodiment, provided herein is a method of treating, preventing, or managing one or more symptoms of relapsed/refractory multiple myeloma in a subject with renal impairment, comprising administering to the subject pomalidomide.

In yet another embodiment, provided herein is a method of treating, preventing, or managing one or more symptoms of multiple myeloma in a subject with renal impairment, comprising administering to the subject pomalidomide; wherein the subject has exhausted lenalidomide and bortezomib therapy.

In yet another embodiment, provided herein is a method of treating, preventing, or managing one or more symptoms of relapsed/refractory multiple myeloma in a subject with renal impairment, comprising administering to the subject pomalidomide; wherein the subject has exhausted lenalidomide and bortezomib therapy.

In yet another embodiment, provided herein is a method of treating, preventing, or managing one or more symptoms of multiple myeloma in a subject with renal impairment, comprising administering to the subject pomalidomide; wherein the subject has at least two prior threapies (e.g., lenalidomide and bortezomib) for treating myeloma treatments.

In still another embodiment, provided herein is a method of treating, preventing, or managing one or more symptoms of relapsed/refractory multiple myeloma in a subject with renal impairment, comprising administering to the subject pomalidomide; wherein the subject has at least two prior threapies (e.g., lenalidomide and bortezomib) for treating myeloma treatments.

Also provided herein is a method of treating, preventing, or managing one or more symptoms of a disease in a subject with renal impairment, comprising administering to the subject a therapeutically effective amount of pomalidomide and dexamethasone.

In one embodiment, provided herein is a method of treating, preventing, or managing one or more symptoms of cancer in a subject with renal impairment, comprising administering to the subject a therapeutically effective amount of pomalidomide and dexamethasone.

In another embodiment, provided herein is a method of treating, preventing, or managing one or more symptoms of hematological cancer in a subject with renal impairment, comprising administering to the subject a therapeutically effective amount of pomalidomide and dexamethasone.

In yet another embodiment, provided herein is a method of treating, preventing, or managing one or more symptoms of multiple myeloma in a subject with renal impairment, comprising administering to the subject a therapeutically effective amount of pomalidomide and dexamethasone.

In yet another embodiment, provided herein is a method of treating, preventing, or managing one or more symptoms of relapsed/refractory multiple myeloma in a subject with renal impairment, comprising administering to the subject a therapeutically effective amount of pomalidomide and dexamethasone.

In yet another embodiment, provided herein is a method of treating, preventing, or managing one or more symptoms of multiple myeloma in a subject with renal impairment, comprising administering to the subject a therapeutically effective amount of pomalidomide and dexamethasone; wherein the subject has exhausted lenalidomide and bortezomib therapy.

In yet another embodiment, provided herein is a method of treating, preventing, or managing one or more symptoms of relapsed/refractory multiple myeloma in a subject with renal impairment, comprising administering to the subject a therapeutically effective amount of pomalidomide and dexamethasone; wherein the subject has exhausted lenalidomide and bortezomib therapy.

In yet another embodiment, provided herein is a method of treating, preventing, or managing one or more symptoms of multiple myeloma in a subject with renal impairment, comprising administering to the subject a therapeutically effective amount of pomalidomide and dexamethasone; wherein the subject has at least two prior threapies (e.g., lenalidomide and bortezomib) for treating myeloma treatments.

In still another embodiment, provided herein is a method of treating, preventing, or managing one or more symptoms of relapsed/refractory multiple myeloma in a subject with renal impairment, comprising administering to the subject a therapeutically effective amount of pomalidomide and dexamethasone; wherein the subject has at least two prior threapies (e.g., lenalidomide and bortezomib) for treating myeloma treatments.

Furthermore, provided herein is a method of treating, preventing, or managing one or more symptoms of a disease in a subject with renal impairment, comprising administering to the subject a therapeutically effective amount of pomalidomide and a subtherapeutically effective amount of dexamethasone.

In one embodiment, provided herein is a method of treating, preventing, or managing one or more symptoms of cancer in a subject with renal impairment, comprising administering to the subject a therapeutically effective amount of pomalidomide and a subtherapeutically effective amount of dexamethasone.

In another embodiment, provided herein is a method of treating, preventing, or managing one or more symptoms of hematological cancer in a subject with renal impairment, comprising administering to the subject a therapeutically effective amount of pomalidomide and a subtherapeutically effective amount of dexamethasone.

In yet another embodiment, provided herein is a method of treating, preventing, or managing one or more symptoms of multiple myeloma in a subject with renal impairment, comprising administering to the subject a therapeutically effective amount of pomalidomide and a subtherapeutically effective amount of dexamethasone.

In yet another embodiment, provided herein is a method of treating, preventing, or managing one or more symptoms of relapsed/refractory multiple myeloma in a subject with renal impairment, comprising administering to the subject a therapeutically effective amount of pomalidomide and a subtherapeutically effective amount of dexamethasone.

In yet another embodiment, provided herein is a method of treating, preventing, or managing one or more symptoms of multiple myeloma in a subject with renal impairment, comprising administering to the subject a therapeutically effective amount of pomalidomide and a subtherapeutically effective amount of dexamethasone; wherein the subject has exhausted lenalidomide and bortezomib therapy.

In yet another embodiment, provided herein is a method of treating, preventing, or managing one or more symptoms of relapsed/refractory multiple myeloma in a subject with renal impairment, comprising administering to the subject a therapeutically effective amount of pomalidomide and a subtherapeutically effective amount of dexamethasone; wherein the subject has exhausted lenalidomide and bortezomib therapy.

In yet another embodiment, provided herein is a method of treating, preventing, or managing one or more symptoms of multiple myeloma in a subject with renal impairment, comprising administering to the subject a therapeutically effective amount of pomalidomide and a subtherapeutically effective amount of dexamethasone; wherein the subject has at least two prior threapies (e.g., lenalidomide and bortezomib) for treating myeloma treatments.

In still another embodiment, provided herein is a method of treating, preventing, or managing one or more symptoms of relapsed/refractory multiple myeloma in a subject with renal impairment, comprising administering to the subject a therapeutically effective amount of pomalidomide and a subtherapeutically effective amount of dexamethasone; wherein the subject has at least two prior threapies (e.g., lenalidomide and bortezomib) for treating myeloma treatments.

Additionally, provided herein is a method of inhibiting the growth of a cell, comprising contacting the cell with pomalidomide and dexamethasone.

Provided herein is a method of inhibiting the growth of a cell, comprising contacting the cell with pomalidomide and a subtherapeutically effective amount of dexamethasone.

### 5. DETAILED DESCRIPTION

### 5.1 Definitions

Generally, the nomenclature used herein and the laboratory procedures in medicinal chemistry, biochemistry, biology, and pharmacology described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, and mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself. In certain embodiments, the terms refer to minimizing the spread or worsening of a disorder, disease, or condition resulting from the administration of one or more prophylactic or therapeutic agents to a subject with such a disorder, disease, or condition. In certain embodiments, the terms refer to the administration of a therapeutic agent after the onset of one or more symptoms of a particular disorder, disease, or condition.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition. In certain embodiments, the terms refer to the treatment with or administration of a therapeutic agent prior to the onset of one or more symptoms of the disorder, disease, or condition, particularly to a subject at risk of such a disorder, disease, or condition. The terms encompass the inhibition or reduction of one or more symptoms of a particular disorder, disease, or condition. Subjects with familial history of a disorder, disease, or condition in particular are candidates for preventive regimens in certain embodiments. In addition, subjects who have a history of recurring symptoms are also potential candidates for the prevention. In this regard, the term “prevention” may be interchangeably used with the term “prophylactic treatment.”

The terms “manage,” “managing,” and “management” refer to preventing or slowing the progression, spread, or worsening of a disorder, disease, or condition, or of one or more symptoms thereof. Often, the beneficial effects that a subject derives from a prophylactic and/or therapeutic agent do not result in a cure of the disorder, disease, or condition. In this regard, the term “managing” encompasses treating a subject who had suffered from a particular disorder, disease, or condition in an attempt to prevent or minimize the recurrence of the disorder, disease, or condition, or lengthening the time during which the disorder, disease, or condition remains in remission.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The terms “tumor,” “neoplasm,” and “neoplastic disorder or disease” are used interchangeably herein and are meant to refer to unwanted cell proliferation of one or more subset of cells in a multicellular organism resulting in harm (i.e., discomfort or decreased life expectancy) to the multicellular organisms. In certain embodiments, a tumor is benign (non-invasive) or malignant (invasive).

The term “cancer” is meant to refer to a malignant neoplasm, which is characterized by uncontrolled cell proliferation, where cells have lost their normal regulatory controls that would otherwise govern the rate of cell growth. These unregulated, dividing cells can spread throughout the body and invade normal tissues in a process referred to as “metastasis.”

The terms “hematological malignancy” and “hematological cancer” are used interchangeably herein and refer to cancer of the body's blood-forming and immune system—the bone marrow and lymphatic tissue. Examples of hematological malignancies include, for instance, myelodysplasia, lymphomas, leukemias, lymphomas (non-Hodgkin's lymphoma), Hodgkin's disease (also called Hodgkin's lymphoma), and myeloma, such as acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma (MM).

The term “therapeutically effective amount” of a therapeutic agent refers to the amount of the therapeutic agent that, when administered, is sufficient to prevent the development of, or alleviate to some extent, one or more of the symptoms of a disorder, disease, or condition being treated. The term also refers to the amount of the therapeutic agent that is sufficient to elicit a biological or medical response of a biological molecule (e.g., a protein, enzyme, RNA, or DNA), cell, tissue, system, animal, or human, which is being sought by a researcher, veterinarian, medical doctor, or clinician. Furthermore, a therapeutically effective amount of a therapeutic agent means an amount of a therapeutic agent, alone or in combination with other therapies, which provides a therapeutic benefit in the treatment or management of a disorder, disease, or condition. The term encompasses an amount of a therapeutic agent that improves overall therapy, reduces, or avoids symptoms or causes of a disorder, disease, or condition, or enhances the therapeutic efficacy of another therapeutic agent.

The terms “low-dose,” subtherapeutic amount,” and “subtherapeutically effective amount” of a therapeutic agent are used interchangeably herein and refer to a dose lower than the amount which is effective when the therapeutic agent is delivered alone (monotherapy). Although less desirable, it is possible that one of the active agents may be used in a supratherapeutic amount, i.e., at a higher dosage in the combination than when used alone. In this embodiment, the other active agent(s) may be used in a therapeutic or subtherapeutic amount.

The term “prophylactically effective amount” of a therapeutic agent refers to the amount of the therapeutic agent that is sufficient to prevent a disorder, disease, or condition, or prevent its recurrence. In certain embodiments, the term “prophylactically effective amount” encompasses an amount that improves overall prophylaxis or enhances the prophylactic efficacy of another prophylactic agent.

The terms “pharmaceutically acceptable carrier,” “pharmaceutically acceptable excipient,” “physiologically acceptable carrier,” and “physiologically acceptable excipient” are used interchangeably herein and refer to a pharmaceutically acceptable material, composition, or vehicle, such as a liquid or solid filler, diluent, solvent, or encapsulating material. In one embodiment, each component is “pharmaceutically acceptable” in the sense of being compatible with other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, Pa., 2005; Handbook of Pharmaceutical Excipients, 7th Edition, Rowe et al., Eds., The Pharmaceutical Press and the American Pharmaceutical Association: 2012; Handbook of Pharmaceutical Additives, 3rd Edition, Ash and Ash Eds., Gower Publishing Company: 2007; and Pharmaceutical Preformulation and Formulation, 2nd Edition, Gibson Ed., CRC Press LLC: Boca Raton, Fla., 2009.

The term “pharmaceutically acceptable salt” refers an acid or base addition salt of a therapeutic agent, such as pomalidomide and dexamethasone. See, Berge et al., J. Pharm. Sci. 1977, 66, 1-19; and “Handbook of Pharmaceutical Salts, Properties, and Use,” Stahl and Wermuth, Ed.; Wiley-VCH and VHCA, Zurich, 2002.

Suitable acids for a pharmaceutically acceptable salt of a therapeutic agent include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, a-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for a pharmaceutically acceptable salt of a therapeutic agent include, but are not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The term “isotopic variant” refers to a therapeutic agent that contains an unnatural proportion of an isotope at one or more of the atoms that constitute such a therapeutic agent. In certain embodiments, an “isotopic variant” of a therapeutic agent contains unnatural proportions of one or more isotopes, including, but not limited to, hydrogen (¹H), deuterium (²H), tritium (³H), carbon-11 (¹¹C) carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I) iodine-125 (¹²⁵) iodine-127 (¹²⁷I) iodine-129 (¹²⁹I) and iodine-131 (¹³¹I) In certain embodiments, an “isotopic variant” of a therapeutic agent is in a stable form, that is, non-radioactive. In certain embodiments, an “isotopic variant” of a therapeutic agent contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I) In certain embodiments, an “isotopic variant” of a therapeutic agent is in an unstable form, that is, radioactive. In certain embodiments, an “isotopic variant” of a therapeutic agent contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I) iodine-125 (¹²⁵I) iodine-129 (¹²⁹I) and iodine-131 (¹³¹I) It will be understood that, in a therapeutic agent, any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a therapeutic agent contains unnatural proportions of deuterium (D).

The term “solvate” refers to a complex or aggregate formed by one or more molecules of a solute, e.g., a therapeutic agent, and one or more molecules of a solvent, which present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The term “pomalidomide” refers to 4-amino-2-(2,6-dioxopiperidin-3-yl)isoindole-1,3-dione or an isotopic variant; or a pharmaceutically acceptable salt, hydrate, or solvent thereof. In one embodiment, pomalidomide is 4-amino-2-(2,6-dioxopiperidin-3-yl)isoindole-1,3-dione. In another embodiment, pomalidomide is an isotopic variant of 4-amino-2-(2,6-dioxopiperidin-3-yl)isoindole-1,3-dione. In yet another embodiment, pomalidomide is a deuterated 4-amino-2-(2,6-dioxopiperidin-3-yl)isoindole-1,3-dione.

The term “dexamethasone” refers to (8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one or an isotopic variant; or a pharmaceutically acceptable salt, hydrate, or solvent thereof. In one embodiment, dexamethasone is (8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one. In another embodiment, dexamethasone is an isotopic variant of (8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one. In yet another embodiment, dexamethasone is a deuterated (8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one.

The term “relapsed” refers to a situation where a subject, which has had a remission of cancer after therapy has a return of cancer cells.

The term “refractory or resistant” refers to a circumstance where a subject, even after intensive treatment, has residual cancer cells in the subject's body.

The term “drug resistance” refers to the condition when a disease does not respond to the treatment of a drug or drugs. Drug resistance can be either intrinsic, which means the disease has never been responsive to the drug or drugs, or it can be acquired, which means the disease ceases responding to a drug or drugs that the disease had previously responded to. In certain embodiments, drug resistance is intrinsic. In certain embodiments, the drug resistance is acquired.

The term “renal impairment” refers to an impaired renal function in a subject. Glomerular filtration rate (GFR) is an indicator of renal function. Renal function can be assessed using a number of criteria, including, but not limited to, serum creatinine levels, urinary creatinine levels, urinary albumin levels, urinary microproteins levels (e.g., retinol binding protein, N-acetyl-β-D-glucosaminidase, and microalbumin), plasma clearance of inulin, creatinine clearance, and proteinurea. In certain embodiments, the renal function of a subject is measured by a creatinine level. In certain embodiments, the renal function of a subject is measured by a serum creatinine level. In certain embodiments, the renal function of a subject is measured by a urinary creatinine level. In certain embodiments, the renal function of a subject is measured by creatinine clearance. In certain embodiments, a renally impaired subject has a creatinine clearance rate of no greater than about 80 mL/min. In certain embodiments, a renally impaired subject has a mild renal impairment, which is characterized by a creatinine clearance rate ranging from about 50 to about 80 mL/min. In certain embodiments, a renally impaired subject has a moderate renal impairment, which is characterized by a creatinine clearance rate ranging from about 30 to about 50 mL/min. In certain embodiments, a renally impaired subject has a severe renal impairment, which is characterized by a creatinine clearance rate of no greater than about 30 mL/min. In certain embodiments, a renally impaired subject requires hemodialysis. Subjects may also be classified as mild, moderate or severe using criteria known in the art (see, e.g., McCullough, Rev. Cardiovasc. Med. 2003; 4(suppl. 1): S2-S6).

The terms “about” and “approximately” are used interchangeably herein and mean an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

### 5.2 Methods of Treatment

In one embodiment, provided herein is a method of treating, preventing, or managing one or more symptoms of a disease in a subject with renal impairment, comprising administering to the subject pomalidomide.

In another embodiment, provided herein is a method of treating, preventing, or managing one or more symptoms of a disease in a subject with renal impairment, comprising administering to the subject a therapeutically effective amount of pomalidomide and dexamethasone. In certain embodiments, the combination of pomalidomide and dexamethasone has a synergetic effect in treating, preventing, or managing one or more symptoms of the disease compared to the administration of pomalidomide and dexamethasone alone.

In yet another embodiment, provided herein is a method of treating, preventing, or managing one or more symptoms of a disease in a subject with renal impairment, comprising administering to the subject pomalidomide and a subtherapeutically effective amount of dexamethasone. In certain embodiments, the combination of pomalidomide and a subtherapeutically effective amount of dexamethasone has a synergetic effect in treating, preventing, or managing one or more symptoms of the disease compared to the administration of pomalidomide and dexamethasone alone.

In certain embodiments, pomalidomide is administered in a therapeutically effective amount. In certain embodiments, pomalidomide is administered in a subtherapeutically effective amount.

In all of the embodiments provided herein, when a renally impaired patient is treated, there is a need for administering to the renally impaired patient a dose of pomalidomide lower than the dose administered to a normal patient (e.g., a patient without renal impairment) because of the decreased ability of the renally impaired patient in eliminating pomalidomide or its metabolites. Thus, in one embodiment, provided herein is a method for treating a renally impaired patient with a dose of pomalidomide lower than the dose administered to a normal patient.

In certain embodiments, pomalidomide is administered in a single dose or divided doses to the subject in the amount ranging from about 0.1 to about 100 mg per day, from about 1 to about 50 mg per day, from about 1 to about 25 mg per day, from about 2 to about 25 mg per day, from about 2 to about 20 mg per day, or from about 2 to about 15 mg per day. In certain embodiments, pomalidomide is administered in a single dose or divided doses to the subject in the amount ranging from about 0.1 to about 100 mg per day. In certain embodiments, pomalidomide is administered in a single dose or divided doses to the subject in the amount ranging from about 1 to about 50 mg per day. In certain embodiments, pomalidomide is administered in a single dose or divided doses to the subject in the amount ranging from about 1 to about 25 mg per day. In certain embodiments, pomalidomide is administered in a single dose or divided doses to the subject in the amount ranging from about 2 to about 25 mg per day. In certain embodiments, pomalidomide is administered in a single dose or divided doses to the subject in the amount ranging from about 2 to about 20 mg per day. In certain embodiments, pomalidomide is administered in a single dose or divided doses to the subject in the amount ranging from about 2 to about 15 mg per day.

In certain embodiments, pomalidomide is administered in a single dose or divided doses to the subject in the amount of about 1, about 2, about 3, about 4, about 5, about 6, about 7, about 8, about 9, about 10, about 11, about 12, about 13, about 14, about 15 mg per day. In certain embodiments, pomalidomide is administered in a single dose or divided doses to the subject in the amount of about 1, about 2, about 3, about 4, or about 5 mg per day.

Depending on the disease to be treated and the subject's condition, pomalidomide can be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. Pomalidomide can be formulated, alone or together, in suitable dosage unit with pharmaceutically acceptable excipients, carriers, adjuvants and vehicles, appropriate for each route of administration. In certain embodiments, pomalidomide is administered orally. In certain embodiments, pomalidomide is administered parenterally. In certain embodiments, pomalidomide is administered intravenously.

Pomalidomide can be delivered as a single dose, such as, e.g., a single bolus injection, or oral tablets or pills; or over time, such as, e.g., continuous infusion over time.

In certain embodiments, the frequency of administration of pomalidomide is in the range of about a daily dose to about a monthly dose. In certain embodiments, the administration of pomalidomide is once a day, twice a day, three times a day, four times a day, once every other day, twice a week, once every week, once every two weeks, once every three weeks, or once every four weeks. In certain embodiments, pomalidomide is administered once a day (QD), twice a day (BID), three times a day (TIB), or four times a day (QIB). In certain embodiments, pomalidomide is administered once a day. In certain embodiments, pomalidomide is administered twice a day. In certain embodiments, pomalidomide is administered three times a day. In certain embodiments, pomalidomide is administered four times a day.

In certain embodiments, pomalidomide is administered daily in a 28 day treatment cycle. In certain embodiments, pomalidomide is administered for 21 days in a 28 day treatment cycle. In certain embodiments, pomalidomide is administered daily on days 1 to 21 in a 28 day treatment cycle.

In certain embodiments, pomalidomide is administered with food.

In certain embodiments, pomalidomide is administered on an empty stomach. In certain embodiments, pomalidomide is administered at least about one hour before eating or at least about two hours after eating. In certain embodiments, pomalidomide is administered at least about one hour before eating. In certain embodiments, pomalidomide is administered at least about two hours after eating.

In certain embodiments, dexamethasone is administered in a therapeutically effective amount. In certain embodiments, dexamethasone is administered in a subtherapeutically effective amount. In certain embodiments, it was unexpected and surprising to find that low dose dexamethasone was more effective than high-dose dexamethasone when combined with pomalidomide in treating cancer (e.g., multiple myeloma).

In certain embodiments, dexamethasone is administered in a single dose or divided doses to the subject in the amount ranging from about 1 to about 500 mg per week, from about 5 to about 250 mg per week, from about 10 to about 100 mg per week, or from about 10 to about 50 mg per week. In certain embodiments, dexamethasone is administered in a single dose or divided doses to the subject in the amount ranging from about 1 to about 500 mg per week. In certain embodiments, dexamethasone is administered in a single dose or divided doses to the subject in the amount ranging from about 5 to about 250 mg per week. In certain embodiments, dexamethasone is administered in a single dose or divided doses to the subject in the amount ranging from about 10 to about 100 mg per week. In certain embodiments, dexamethasone is administered in a single dose or divided doses to the subject in the amount ranging from about 10 to about 50 mg per week. In certain embodiments, dexamethasone is administered in a single dose or divided doses to the subject in the amount of about 10, about 15, about 20, about 25, about 30, about 35, about 40, about 45, or about 50 mg per week. In certain embodiments, dexamethasone is administered in a single dose or divided doses to the subject in the amount of about 20, about 30, or about 40 mg per week.

Depending on the disease to be treated and the subject's condition, dexamethasone can be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. Dexamethasone can be formulated, alone or together, in suitable dosage unit with pharmaceutically acceptable excipients, carriers, adjuvants and vehicles, appropriate for each route of administration. In certain embodiments, dexamethasone is administered orally. In certain embodiments, dexamethasone is administered parenterally. In certain embodiments, dexamethasone is administered intravenously. In certain embodiments, dexamethasone is administered topically.

Dexamethasone can be delivered as a single dose, such as, e.g., a single bolus injection, or oral tablets or pills; or over time, such as, e.g., continuous infusion over time.

In certain embodiments, the frequency of administration of dexamethasone is in the range of about a daily dose to about a monthly dose. In certain embodiments, the administration of dexamethasone is once a day, twice a day, three times a day, four times a day, once every other day, twice a week, once every week, once every two weeks, once every three weeks, or once every four weeks. In certain embodiments, dexamethasone is administered once a day (QD), twice a day (BID), three times a day (TIB), or four times a day (QIB). In certain embodiments, dexamethasone is administered once a day. In certain embodiments, dexamethasone is administered twice a day. In certain embodiments, dexamethasone is administered three times a day. In certain embodiments, dexamethasone is administered four times a day. In certain embodiments, dexamethasone is administered once a week.

In certain embodiments, dexamethasone is administered every week in a 28 day treatment cycle. In certain embodiments, dexamethasone is administered for four days in a 28 day treatment cycle. In certain embodiments, dexamethasone is administered daily on days 1, 8, 15, and 22 in a 28 day treatment cycle. In certain embodiments, dexamethasone is administered for eight days in a 28 day treatment cycle. In certain embodiments, dexamethasone is administered for twelve days in a 28 day treatment cycle. In certain embodiments, dexamethasone is administered daily on days 1 to 4, 9 to 12, and 17 to 20 in a 28 day treatment cycle.

In certain embodiments, dexamethasone is administered with food.

In certain embodiments, dexamethasone is administered on an empty stomach. In certain embodiments, dexamethasone is administered at least about one hour before eating or at least about two hours after eating. In certain embodiments, dexamethasone is administered at least about one hour before eating. In certain embodiments, dexamethasone is administered at least about two hours after eating.

In certain embodiments, the combination regimen (i.e., the combination of pomalidomide and dexamethasone) is administered to the subject over an extended period of time, ranging from 1 day to about 12 months, from 2 days to about 6 months, from 3 days to about 5 months, from 3 days to about 4 months, from 3 days to about 12 weeks, from 3 days to about 10 weeks, from 3 days to about 8 weeks, from 3 days to about 6 weeks, from 3 days to about 5 weeks, from 3 days to about 4 weeks, from 3 days to about 3 weeks, from 3 days to about 2 weeks, or from 3 days to about 10 days.

In certain embodiments, the combination regimen is cyclically administered to the subject. Cycling therapy involves the administration of the combination regimen for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

Consequently, in one embodiment, the combination regimen is administered daily for one week, two weeks, three weeks, four weeks, five weeks, six weeks, eight weeks, ten weeks, fifteen weeks, or twenty weeks, followed by a rest period of about 1 day to about ten weeks. In certain embodiments, the combination regimen is administered daily for one week, two weeks, three weeks, four weeks, five weeks, or six weeks with a rest period of 1, 3, 5, 7, 9, 12, 14, 16, 18, 20, 22, 24, 26, 28, 29 or 30 days. In certain embodiments, the rest period is 7 days. In certain embodiments, the rest period is 14 days. In certain embodiments, the rest period is 28 days. In one embodiment, the rest period is a period that is sufficient for bone marrow recovery. The frequency, number and length of dosing cycles can be increased or decreased.

As used herein, the term “combination regimen” includes the use of more than one therapies (e.g., one or more prophylactic and/or therapeutic agents). However, the use of the term “combination regimen” does not restrict the order in which therapies (e.g., prophylactic and/or therapeutic agents) are administered to the subject. A first therapy (e.g., a prophylactic or therapeutic agent such as pomalidomide) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as dexamethasone) to the subject. Triple therapy is also contemplated herein (e.g., a platinum agent as a third therapy).

In certain embodiments, pomalidomide is administered to the subject prior to the administration of dexamethasone. In certain embodiments, pomalidomide is administered to the subject about 2 days, about 1 day, about 12 hrs, about 6 hrs, about 4 hrs, about 2 hrs, about 60 min, about 30 min, about 10 min before the administration of dexamethasone.

In certain embodiments, pomalidomide is administered to the subject concurrently with the administration of dexamethasone.

In certain embodiments, pomalidomide is administered to the subject after the administration of dexamethasone. In certain embodiments, pomalidomide is administered to the subject about 2 days, about 1 day, about 12 hrs, about 6 hrs, about 4 hrs, about 2 hrs, about 60 min, about 30 min, about 10 min after the administration of dexamethasone.

In certain embodiments, pomalidomide is administered for 21 days and dexamethasone is administered every week in a 28 day treatment cycle. In certain embodiments, pomalidomide is administered for 21 days and dexamethasone is administered for four days in a 28 day treatment cycle. In certain embodiments, pomalidomide is administered daily on days 1 to 21 and dexamethasone is administered daily on days 1, 8, 15, and 22 in a 28 day treatment cycle.

In certain embodiments, pomalidomide is administered in a therapeutically effective amount for 21 days and dexamethasone is administered in a subtherapeutically effective amount every week in a 28 day treatment cycle. In certain embodiments, pomalidomide is administered in a therapeutically effective amount for 21 days and dexamethasone is administered in a subtherapeutically effective amount for four days in a 28 day treatment cycle. In certain embodiments, pomalidomide is administered in a therapeutically effective amount daily on days 1 to 21 and dexamethasone is administered in a subtherapeutically effective amount daily on days 1, 8, 15, and 22 in a 28 day treatment cycle.

In certain embodiments, pomalidomide is administered in the amount ranging from about 2 to about 15 mg per day for 21 days in a 28 day treatment cycle. In certain embodiments, pomalidomide is administered in the amount ranging from about 2 to about 15 mg per day on days 1 to 21.

In certain embodiments, pomalidomide is administered in the amount ranging from about 2 to about 15 mg per day for 21 days and dexamethasone is administered in the amount ranging from about 10 to about 50 mg every week in a 28 day treatment cycle. In certain embodiments, pomalidomide is administered in the amount ranging from about 2 to about 15 mg per day for 21 days and dexamethasone is administered in the amount ranging from about 10 to about 50 mg per day for four days in a 28 day treatment cycle. In certain embodiments, pomalidomide is administered in the amount ranging from about 2 to about 15 mg per day on days 1 to 21 and dexamethasone is administered in the amount ranging from about 10 to about 50 mg per day on days 1, 8, 15, and 22 in a 28 day treatment cycle.

In certain embodiments, pomalidomide is administered in the amount of about 2 mg or about 4 mg per day for 21 days in a 28 day treatment cycle. In certain embodiments, pomalidomide is administered in the amount of about 2 mg or about 4 mg per day on days 1 to 21 in a 28 day treatment cycle.

In certain embodiments, pomalidomide is administered in the amount of about 2 mg or about 4 mg per day for 21 days and dexamethasone is administered in the amount of about 20 mg or about 40 mg every week in a 28 day treatment cycle. In certain embodiments, pomalidomide is administered in the amount of about 2 mg or about 4 mg per day for 21 days and dexamethasone is administered in the amount of about 20 mg or about 40 mg per day for four days in a 28 day treatment cycle. In certain embodiments, pomalidomide is administered in the amount of about 2 mg or about 4 mg per day on days 1 to 21 and dexamethasone is administered in the amount of about 20 or about 40 mg per day on days 1, 8, 15, and 22 in a 28 day treatment cycle.

The combination regimen (i.e., the combination of pomalidomide and dexamethasone) can be administered repetitively if necessary, for example, until the subject being treated experiences stable disease or regression, or until the subject experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 2000, 92, 205-216. Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

In certain embodiments, the subject is a mammal. In certain embodiments, the mammal is a human.

In certain embodiments, the subject to be treated with one of the methods provided herein has not been treated with anticancer therapy. In certain embodiments, the subject to be treated with one of the methods provided herein has been treated with anticancer therapy.

In certain embodiments, the disease is one related to PDE4, TNFα, cAMP, and/or angiogenesis, including, but not limited to, inflammatory diseases, pulmonary diseases, autoimmune diseases, and immunological diseases.

In certain embodiments, the disease is complex regional pain syndrome (“CRPS”), macular degeneration (“MD”), a skin disease, a pulmonary disorder, an asbestos-related disorder, a parasitic disease, an immunodeficiency disorder, a CNS disorder, CNS injury, atherosclerosis, a dysfunctional sleep, hemoglobinopathy, anemia, tuberculosis, a PDE4/TNFα related disorder, or an infectious disease.

In certain embodiments, the disease is an inflammatory, viral, genetic, allergic, skin, or autoimmune disease. In certain embodiments, the disease is an inflammatory disease.

In certain embodiments, the disease is arthritis, HIV, hepatitis, acne, adult respiratory distress syndrome, a bone resorption disease, a chronic pulmonary inflammatory disease, dermatitis, dematomyositis, cystic fibrosis, Lichen Planus, septic shock, sepsis, endotoxic shock, hemodynamic shock, sepsis syndrome, post ischemic reperfusion injury, meningitis, psoriasis, fibrotic disease, cachexia, graft versus host disease, graft rejection, autoimmune disease, rheumatoid spondylitis, Behcet's disease, dermatitis, Crohn's disease, ulcerative colitis, inflammatory-bowel disease, rosacea, multiple sclerosis, systemic lupus erythrematosus, ENL in leprosy, sacoidosis, radiation damage, asthma, uveitis, or hyperoxic alveolar injury.

In certain embodiments, the disease is lupus erythrematosus. In certain embodiments, the disease is systemic lupus erythematosus (SLE), cutaneous lupus erythematosus (CLE), or drug-induced lupus.

In certain embodiments, the disease is an immune-related disease. In certain embodiments, the disease is Sjögren syndrome, ANCA-induced vasculitis, anti-phospholipid syndrome, myasthenia gravis, Addison's disease, alopecia areata, ankylosing spondylitis, antiphospholipid antibody syndrome, antiphospholipid syndrome (primary or secondary), asthma, autoimmune gastritis, autoimmune hemolytic anemia, autoimmune hepatitis, autoimmune inner ear disease, autoimmune lymphoproliferative disease, autoimmune thrombocytopenic purpura, Balo disease, Behcet's disease, bullous pemphigoid, cardiomyopathy, celiac disease, Chagas disease, chronic inflammatory demyelinating polyneuropathy, cicatrical pemphigoid (e.g., mucous membrane pemphigoid), cold agglutinin disease, degos disease, dermatitis hepatiformis, essential mixed cryoglobulinemia, Goodpasture's syndrome, Graves' disease, Guillain-Barre syndrome, Hashimoto's thyroiditis (Hashimoto's disease; autoimmune thyroditis), idiopathic pulmonary fibrosis, idiopathic thrombocytopenia purpura, IgA nephropathy, juvenile arthritis, lichen planus, Méniére disease, mixed connective tissue disease, morephea, narcolepsy, neuromyotonia, pediatric autoimmune neuropsychiatric disorders (PANDAs), pemphigus vulgaris, pernicious anemia, polyarteritis nodosa, polychondritis, polymyalgia rheumatica, primary agammaglobulinemia, primary biliary cirrhosis, Raynaud disease (Raynaud phenomenon), Reiter's syndrome, relapsing polychondritis, rheumatic fever, Sjogren's syndrome, stiff-person syndrome (Moersch-Woltmann syndrome), Takayasu's arteritis, temporal arteritis (giant cell arteritis), uveitis, vasculitis (e.g., vasculitis not associated with lupus erythematosus), vitiligo, or Wegener's granulomatosis.

In certain embodiments, the disease is psoriasis or plaque psoriasis. In certain embodiments, the disease is arthritis, psoriatic arthritis, rheumatoid arthritis, osteoarthritis, or acute gouty arthritis. In certain embodiments, the disease is ankylosing spondylitis. In certain embodiments, the disease is a skin disease, acne, dermatitis, dermatomyositis, atopic dermatitis, or contact dermatitis. In certain embodiments, the disease is sarcoidosis or chronic cutaneous sarcoidosis. In certain embodiments, the disease is uveitis, rosacea, Lichen Planus, Behcet's disease, psoriasis, psoriatic arthritis, rheumatoid arthritis, Behcet's disease, or ankylosing spondylitis.

In certain embodiments, the disease is cancer or a precancerous condition. In certain embodiments, the disease is cancer. Examples of cancer and precancerous conditions include, but are not limited to, those described in U.S. Pat. Nos. 6,962,940 and 7,893,101; the disclosure of each of which is incorporated herein by reference in its entirety.

In certain embodiments, the disease is a solid tumor. In certain embodiments, the disease is skin cancer, melanoma, lymph node cancer, breast cancer, cervix cancer, uterus cancer, gastrointestinal tract cancer, stomach cancer, endrometrium cancer, esophagus cancer, lung cancer, ovary cancer, prostate cancer, colon cancer, rectum cancer, mouth cancer, brain cancer, head and neck cancer, eye cancer, throat cancer, mouth cancer, chest cancer, lymph node cancer, testes cancer, kidney cancer, pancreas cancer, bone cancer, spleen cancer, liver cancer, bladder cancer, larynx cancer, nasal cancer, or AIDS-related cancer.

In certain embodiments, the disease is hematological cancer or blood borne tumor. In certain embodiments, the disease is myeloma. In certain embodiments, the disease is multiple myeloma. In certain embodiments, the disease is acute and chronic leukemias, for example, lymphoblastic, myelogenous, lymphocytic, and myelocytic leukemias. In certain embodiments, the disease is lymphoma.

In certain embodiments, the disease is advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, chronic lymphocytic leukemia (CLL), Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma (localized melanoma, including, but not limited to, ocular melanoma), malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the disease is scleroderma. In certain embodiments, the scleroderma is localized, systemic, limited, or diffuse scleroderma.

In certain embodiments, the systemic scleroderma comprises CREST syndrome (Calcinosis, Raynaud's syndrome, esophagaeal dysfunction or dysmotility, sclerodactyly, telangiectasia). Scleroderma is also known as systemic sclerosis or progressive systemic sclerosis. In certain embodiments, the disease is Raynaud's disease or syndrome. In certain embodiments, systemic sclerosis comprises scleroderma lung disease, scleroderma renal crisis, cardiac manifestations, muscular weakness (including fatigue or limited CREST), gastrointestinal dysmotility and spasm, and abnormalities in the central, peripheral and autonomic nervous system (including carpal tunnel syndrome followed by trigeminal neuralgia).

In certain embodiments, the limited scleroderma is limited to the hands, the face, neck, or combinations thereof.

In certain embodiments, the diffuse scleroderma comprises skin tightening and also occurs above the wrists (or elbows). In certain embodiments, the diffuse systemic sclerosis is sine scleroderma, comprising internal organ fibrosis, but no skin tightening, or familial progressive systemic sclerosis.

In certain embodiments, the scleroderma is not associated with wasting, such as disease-related wasting.

In certain embodiments, the cancer is metastatic. In certain embodiments, the cancer is relapsed or refractory. In certain embodiments, the cancer is resistance to chemotherapy or radiation.

In certain embodiments, the disease is chronic lymphocytic leukemia, chronic myelocytic leukemia, acute lymphoblastic leukemia, acute myelogenous leukemia, or acute myeloblastic leukemia, including a leukemia that is relapsed, refractory, or resistant.

The term “leukemia” refers malignant neoplasms of the blood-forming tissues. The leukemia includes, but is not limited to, chronic lymphocytic leukemia, chronic myelocytic leukemia, acute lymphoblastic leukemia, acute myelogenous leukemia, and acute myeloblastic leukemia. In certain embodiments, the leukemia is relapsed, refractory or resistant to conventional therapy.

In certain embodiments, the disease is a lymphoma, including non-Hodgkin's lymphoma (NHL). The term “lymphoma” refers a heterogeneous group of neoplasms arising in the reticuloendothelial and lymphatic systems. “NHL” refers to malignant monoclonal proliferation of lymphoid cells in sites of the immune system, including lymph nodes, bone marrow, spleen, liver and gastrointestinal tract. Examples of NHL include, but are not limited to, mantle cell lymphoma (MCL), lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), follicular lymphoma, and any type of the mantle cell lymphomas that can be seen under the microscope (nodular, diffuse, blastic and mentle zone lymphoma).

Examples of skin diseases include, but are not limited to, those described in U.S. App. Publ. No. 2005/0214328, the disclosure of which is incorporated herein by reference in its entirety. Specific examples include, but are not limited to, keratoses and related symptoms, skin diseases or disorders characterized with overgrowths of the epidermis, acne, and wrinkles.

As used herein, the term “keratosis” refers to any lesion on the epidermis marked by the presence of circumscribed overgrowths of the horny layer, including, but not limited to, actinic keratosis, seborrheic keratosis, keratoacanthoma, keratosis follicularis (Darier disease), inverted follicular keratosis, palmoplantar keratoderma (PPK, keratosis palmaris et plantaris), keratosis pilaris, and stucco keratosis. The term “actinic keratosis” also refers to senile keratosis, keratosis senilis, verruca senilis, plana senilis, solar keratosis, keratoderma or keratoma. The term “seborrheic keratosis” also refers to seborrheic wart, senile wart, or basal cell papilloma. Keratosis is characterized by one or more of the following symptoms: rough appearing, scaly, erythematous papules, plaques, spicules or nodules on exposed surfaces (e.g., face, hands, ears, neck, legs and thorax), excrescences of keratin referred to as cutaneous horns, hyperkeratosis, telangiectasias, elastosis, pigmented lentigines, acanthosis, parakeratosis, dyskeratoses, papillomatosis, hyperpigmentation of the basal cells, cellular atypia, mitotic figures, abnormal cell-cell adhesion, dense inflammatory infiltrates and small prevalence of squamous cell carcinomas.

Examples of skin diseases or disorders characterized with overgrowths of the epidermis include, but are not limited to, any conditions, diseases or disorders marked by the presence of overgrowths of the epidermis, including, but not limited to, infections associated with papilloma virus, arsenical keratoses, sign of Leser-Trélat, warty dyskeratoma (WD), trichostasis spinulosa (TS), erythrokeratodermia variabilis (EKV), ichthyosis fetalis (harlequin ichthyosis), knuckle pads, cutaneous melanoacanthoma, porokeratosis, psoriasis, squamous cell carcinoma, confluent and reticulated papillomatosis (CRP), acrochordons, cutaneous horn, cowden disease (multiple hamartoma syndrome), dermatosis papulosa nigra (DPN), epidermal nevus syndrome (ENS), ichthyosis vulgaris, molluscum contagiosum, prurigo nodularis, and acanthosis nigricans (AN).

The methods provided herein encompass treating a subject regardless of patient's age, although some diseases or disorders are more common in certain age groups. Further provided herein is a method for treating a subject who has undergone surgery in an attempt to treat the disease or condition at issue, as well as the one who has not. Because the subjects with cancer have heterogeneous clinical manifestations and varying clinical outcomes, the treatment given to a particular subject may vary, depending on his/her prognosis.

In yet another embodiment, provided herein is a method of inhibiting the growth of a cell, comprising contacting the cell with pomalidomide and dexamethasone.

In yet another embodiment, provided herein is a method of inhibiting the growth of a cell, comprising contacting the cell with pomalidomide and a subtherapeutically effective amount of dexamethasone.

In still another embodiment, provided herein is a method of inhibiting the growth of a cell, comprising contacting the cell with a therapeutically effective amount pomalidomide and a subtherapeutically effective amount of dexamethasone.

In certain embodiments, the cell is a mammalian cell. In certain embodiments, the mammal is a human cell. In certain embodiments, the cell is a tumor cell. In certain embodiments, the cell is mammalian tumor cell. In certain embodiments, the cell is a human tumor cell. In certain embodiments, the cell is a cancerous cell. In certain embodiments, the cell is mammalian cancerous cell. In certain embodiments, the cell is a human cancerous cell.

In certain embodiments, the cancerous cell is a cell of bladder cancer, breast cancer, cervical cancer, colon cancer (e.g., colorectal cancer), endometrial cancer, esophageal cancer, gastric cancer, glioma (e.g., glioblastoma), head and neck cancer, liver cancer, lung cancer (e.g., small cell and non-small cell lung cancers), melanoma, myeloma, neuroblastoma, ovarian cancer, pancreatic cancer, prostate cancer, renal cancer, sarcoma (e.g., osteosarcoma), skin cancer (e.g., squamous cell carcinoma), stomach cancer, testicular cancer, thyroid cancer, or uterine cancer.

In certain embodiments, the cell is treated by contacting with pomalidomide prior to contacting with dexamethasone. In certain embodiments, the cell is treated with pomalidomide, about 2 days, about 1 day, about 12 hrs, about 6 hrs, about 4 hrs, about 2 hrs, about 60 min, about 30 min, or about 10 min before with dexamethasone.

In certain embodiments, the cell is treated by contacting the cell with pomalidomide and dexamethasone concurrently.

In certain embodiments, the cell is treated by contacting with pomalidomide after contacting with dexamethasone. In certain embodiments, the cell is treated with pomalidomide, about 2 days, about 1 day, about 12 hrs, about 6 hrs, about 4 hrs, about 2 hrs, about 60 min, about 30 min, or about 10 min after with dexamethasone.

The inhibition of cell growth can be gauged by, e.g., counting the number of cells contacted with compounds of interest, comparing the cell proliferation with otherwise identical cells not contacted with the compounds, or determining the size of the tumor that encompasses the cells. The number of cells, as well as the size of the cells, can be readily assessed using any method known in the art (e.g., trypan blue exclusion and cell counting, measuring incorporation of ³H-thymidine into nascent DNA in a cell).

### 5.3 Combination Therapy with an Additional Therapeutic Agent

In one embodiment, the methods provided herein each independently further comprise administering an additional therapeutic agent. It is believed that certain combinations work synergistically in the treatment of a particular disease. The additional therapeutic agent can also work to alleviate adverse effects.

In certain embodiments, the additional therapeutic agent is a large molecule (e.g., a protein). In certain embodiments, the additional therapeutic agent is a small molecule (e.g., a synthetic inorganic, organometallic, or organic molecule).

Examples of large therapeutic agents include, but are not limited to, hematopoietic growth factors, cytokines, and monoclonal and polyclonal antibodies. In certain embodiments, the large therapeutic agent is a biological molecule, such as a naturally occurring or artificially made protein, including those proteins that stimulate the survival and/or proliferation of hematopoietic precursor cells and immunologically active poietic cells in vitro or in vivo. In certain embodiments, the additional therapeutic agent is an interleukin, IL-2 recombinant IL-II (“rIL2”), canarypox IL-2, IL-10, IL-12, IL-18, interferon, interferon alfa-2a, interferon alfa-2b, interferon alfa-n1, interferon alfa-n3, interferon beta-Ia, interferon gamma-I b, GM-CF, GM-CSF, GC-CSF, BCG, a cancer antibody, or EPO. In certain embodiments, the additional therapeutic agent is filgrastim (NEUPOGEN®, Amgen, Thousand Oaks, Calif.), sargramostim (LEUKINE®, Immunex, Seattle, Wash.), or recombinant EPO (EPGEN®, Amgen, Thousand Oaks, Calif.).

In certain embodiments, the additional therapeutic agent is an inhibitor of an ActRII receptor or activin-ActRII inhibitor. In certain embodiments, the additional therapeutic agent is an ActRIIA inhibitor or ActRIIB inhibitor. Inhibitors of ActRII receptors can be polypeptides comprising activin-binding domains of ActRII. In certain embodiments, the activin-binding domain comprising polypeptides are linked to an Fc portion of an antibody (i.e., a conjugate comprising an activin-binding domain comprising polypeptide of an ActRII receptor and an Fc portion of an antibody is generated). In certain embodiments, the activin-binding domain is linked to an Fc portion of an antibody via a linker, e.g., a peptide linker. Examples of such non-antibody proteins selected for activin or ActRIIA binding and methods for design and selection of the same are found in WO/2002/088171, WO/2006/055689, WO/2002/032925, WO/2005/037989, US 2003/0133939, and US 2005/0238646, the disclosure of each of which is incorporated herein by reference in its entirety. In one embodiment, the additional therapeutic agent is ACE-11. In another embodiment, the additional therapeutic agent is ACE-536.

Recombinant and mutated forms of GM-CSF can be prepared as described in U.S. Pat. Nos. 5,391,485; 5,393,870; and 5,229,496; the disclosure of each of which is incorporated herein by reference in its entirety. Recombinant and mutated forms of G-CSF can be prepared as described in U.S. Pat. Nos. 4,810,643; 4,999,291; 5,528,823; and 5,580,755; the disclosure of each of which is incorporated herein by reference in its entirety.

This disclosure encompasses the use of native, naturally occurring, and recombinant proteins. The disclosure further encompasses mutants and derivatives (e.g., modified forms) of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins, such as proteins formed by fusing IgG1 or IgG3 to the protein or active portion of the protein of interest. See, e.g., Penichet et al., J. Immunol. Methods 2001; 248:91-101.

In certain embodiments, the additional therapeutic agent is an antibody. In certain embodiments, the additional therapeutic agent is monoclonal or polyclonal antibody. Examples of antibodies include, but are not limited to, trastuzumab (HERCEPTIN®), rituximab (RITUXAN®),bevacizumab (AVASTIN™), pertuzumab (OMNITARG™), tositumomab (BEXXAR®), edrecolomab (PANOREX®), panitumumab, and G250. In certain embodiments, the additional therapeutic agent is an anti-TNF-α antibody.

In certain embodiments, the additional therapeutic agent is a large molecule administered in the form of an anti-cancer vaccine. In certain embodiments, the additional therapeutic agent is a vaccine that secretes, or causes the secretion of, cytokines such as IL-2, SCF, CXC14 (platelet factor 4), G-CSF, and GM-CSF. See, e.g., Emens et al., Curr. Opinion Mol. Ther. 2001; 3(1):77-84.

In certain embodiments, the additional therapeutic agent is a small molecule. In certain embodiments, the additional therapeutic agent is an anti-cancer agent, antibiotics, immunosuppressive agent, or steroid.

Examples of anti-cancer agents include, but are not limited to, abraxane; ace-11; acivicin; aclarubicin; acodazole hydrochloride; acronine; adozelesin; aldesleukin; altretamine; ambomycin; ametantrone acetate; amrubicin; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; herceptin; hydroxyurea; idarubicin hydrochloride; ifosfamide; ilmofosine; iproplatin; irinotecan; irinotecan hydrochloride; lanreotide acetate; lapatinib; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; romidepsin; safingol; safingol hydrochloride; semustine; simtrazene; sparfosate sodium; sparsomycin; spirogermanium hydrochloride; spiromustine; spiroplatin; stem cell treatments such as PDA-001; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Additional examples of anticancer drugs include, but are not limited to, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; b-FGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim;Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (GENASENSE®); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In certain embodiments, the additional therapeutic agent is proteasome inhibitor. In certain embodiments, the proteasome inhibitor is bortezomib, disulfiram, epigallocatechin-3-gallate, salinosporamide A, carfilzomib, ONX 0912, CEP-18770, or MLN9708.

In certain embodiments, the additional therapeutic agent is HDAC inhibitor. In certain embodiments, the HDAC inhibitor is vorinostat, romidepsin, panobinostat, valproic acid, belinostat, mocetinostat, abexinostat, entinostat, SB939, resminostat, givinostat, CUDC-101, AR-42, CHR-2845, CHR-3996, 4SC-202, CG200745, ACY-1215, sulforaphane, kevetrin, or trichostatin A.

In certain embodiments, the additional therapeutic agent is mitotic inhibitor. In one embodiment, the mitotic inhibitor is taxanes, vinca alkaloids, or colchicines. In certain embodiments, the taxane is paclitaxel (Abraxane) or docetaxel. In certain embodiments, the vinca alkaloid is vinblastine, vincristine, vindesine, or vinorelbine.

In certain embodiments, the additional therapeutic agent is oblimersen (GENASENSE®), remicade, docetaxel, celecoxib, melphalan, steroids, gemcitabine, cisplatinum, temozolomide, etoposide, cyclophosphamide, temodar, carboplatin, procarbazine, gliadel, tamoxifen, topotecan, methotrexate, ARISA®, taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (DOXIL®), paclitaxel, ganciclovir, adriamycin, estramustine sodium phosphate) (EMCYT®), sulindac, and etoposide.

### 5.4 Pharmaceutical Compositions and Dosage Forms

In one embodiment, provided herein are pharmaceutical compositions and dosage forms, which comprise pomalidomide and/or dexamethasone, and one or more excipients.

In certain embodiments, pharmaceutical compositions and dosage forms provided herein also comprise one or more additional therapeutic agents as described herein.

Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal, or transcutaneous administration to a patient. Examples of dosage forms include, but are not limited to, tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms provided herein may vary depending on their use. For example, a dosage form used in the acute treatment of a disease may contain larger amounts of one or more of the active ingredients than a dosage form used in the chronic treatment of the same disease. Similarly, a parenteral dosage form may contain smaller amounts of one or more of the active ingredients than an oral dosage form used to treat the same disease. See, e.g., Remington's Pharmaceutical Sciences, 18th ed., Mack Publishing, Easton Pa. (1990).

Whether a particular excipient is suitable for incorporation into a pharmaceutical composition or dosage form provided herein depends on a variety of factors, including, but not limited to, the route of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, encompassed herein are pharmaceutical compositions and dosage forms that contain little, if any, lactose. As used herein, the term “lactose-free” means that the amount of lactose present, if any, is insufficient to substantially increase the degradation rate of an active ingredient.

Lactose-free compositions provided herein can comprise excipients that are listed, for example, in the U.S. Pharmacopeia (USP) 25-NF20 (2002). In certain embodiments, lactose-free compositions comprise active ingredients, a binder/filler, and a lubricant in pharmaceutically compatible and pharmaceutically acceptable amounts. In certain embodiments, lactose-free dosage forms comprise active ingredients, microcrystalline cellulose, pre-gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, in certain embodiments, provided herein are anhydrous compositions packaged using materials to prevent exposure to water such that they can be included in suitable formulary kits. Examples of suitable packaging include, but are not limited to, hermetically sealed foils, plastics, unit dose containers (e.g., vials), blister packs, and strip packs.

Encompassed herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

### 5.4.1 Oral Dosage Forms

In certain embodiments, pharmaceutical compositions provided herein that are suitable for oral administration are formulated as discrete dosage forms, examples of which include, but are not limited to, tablets (e.g., chewable tablets), caplets, capsules, and liquids (e.g., flavored syrups). Such dosage forms contain predetermined amounts of active ingredients and may be prepared by some known methods of pharmacy. See generally, Remington's Pharmaceutical Sciences, 18th ed., Mack Publishing, Easton Pa. (1990).

In certain embodiments, the oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms may be prepared by some known methods of pharmacy. In certain embodiments, pharmaceutical compositions and dosage forms are prepared by uniformly and intimately admixing the active ingredients with liquid carriers, finely divided solid carriers, or both, and then shaping the product into the desired presentation if necessary.

In certain embodiments, a tablet is prepared by compression or molding. In certain embodiments, compressed tablets are be prepared by compressing in a suitable machine the active ingredients in a free-flowing form, e.g., powder or granules, optionally mixed with an excipient. In certain embodiments, molded tablets are made by molding in a suitable machine a mixture of a powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms provided herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, AVICEL-PH-101, AVICEL-PH-103 AVICEL RC-581, AVICEL-PH-105 (FMC Corporation, American Viscose Division, Avicel Sales, Marcus Hook, Pa.), and mixtures thereof. An specific binder is a mixture of microcrystalline cellulose and sodium carboxymethyl cellulose (e.g., AVICEL RC-581). Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. In certain embodiments, the binder or filler in pharmaceutical compositions provided herein is present in from about 50 to about 99 weight percent of the pharmaceutical composition or dosage form.

Disintegrants are used in the compositions provided herein to provide tablets the ability to disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation. In certain embodiments, the pharmaceutical compositions provided herein comprise from about 0.5 to about 15 weight percent or from about 1 to about 5 weight percent of disintegrant.

Disintegrants that are suitable for use in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that are suitable for use in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, but are not limited to, a syloid silica gel (AEROSIL200, W.R. Grace Co., Baltimore, Md.), a coagulated aerosol of synthetic silica (Degussa Co. of Plano, Tex.), CAB-O-SIL (a pyrogenic silicon dioxide, Cabot Co. of Boston, Mass.), and mixtures thereof. In certain embodiments, if used at all, lubricants are used in an amount of less than about 1 weight percent of the pharmaceutical compositions or dosage forms into which they are incorporated.

### 5.4.2 Delayed Release Dosage Forms

In certain embodiments, the active ingredients provided herein are administered by controlled release means or by delivery devices. Examples include, but are not limited to, those described in U.S. Pat. Nos. 3,845,770; 3,916,899; 3,536,809; 3,598,123; 4,008,719, 5,674,533, 5,059,595, 5,591,767, 5,120,548, 5,073,543, 5,639,476, 5,354,556, and 5,733,566, each of which is incorporated herein by reference in its entirety. In certain embodiments, such dosage forms are be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Encompassed herein are single unit dosage forms suitable for oral administration, including, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 5.4.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Some suitable vehicles that can be used to provide parenteral dosage forms provided herein include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms provided herein. See, e.g., U.S. Pat. No. 5,134,127, the disclosure of which is incorporated herein by reference in its entirety.

### 5.4.4 Topical and Mucosal Dosage Forms

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein depend on the particular tissue to which a given pharmaceutical composition or dosage form will be applied. With that fact in mind, in certain embodiments, the excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Additional examples of such ingredients can be found, e.g., in Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 5.4.5 Kits

Provided herein also are kits which, when used by the medical practitioner, can simplify the administration of appropriate amounts of active ingredients to a subject. In certain embodiments, the kit provided herein includes containers and dosage forms of pomalidomide and dexamethasone in the combination regimens provided herein. In certain embodiments, active ingredients provided herein are not administered to a patient at the same time or by the same route of administration.

In certain embodiments, the kit includes a container comprising dosage forms of pomalidomide and dexamethasone in the combination regimens provided herein, in one or more containers.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### 6. EXAMPLES

The disclosure will be further understood by the following non-limiting examples.

### 6.1 Phase ½ Clinical Study

A multi-center phase ½ clinical study was conducted to evaluate the impact of renal function of patients with relapsed and refractory multiple myeloma (RRMM) on their treatment with pomalidomide (POM) in combination with low-dose dexamethasone (LoDEX). Eligible patients with RRMM who had received ≧2 prior therapies were randomized to treatment with either POM+LoDEX (POM, 4 mg/day for days 1 to 21 of a 28-day cycle; LoDEX, 40 mg/week). At progression, patients receiving POM alone could receive POM+LoDEX at investigator's discretion. Patients were retrospectively categorized into three groups based on calculated baseline creatinine clearance (CrCl) by the Cockcroft-Gault formula: CrCl>60 mL/min; CrCl 45-60 mL/min; and CrCl<45 mL/min. Treatment-emergent adverse events (TEAEs) were defined as any AE occurring or worsening after first treatment with study medication and within 30 days after end of treatment. All patients received aspirin, 81-100 mg/day, or another form of thromboprophylaxis.

A total of 113 patients received POM+LoDEX; the median age of these patients was 64 years (ranging from 34 to 88). Median number of prior therapies was 5 (ranging from 2 to 13). The majority of patients were male (62/113, 54.9%) and had an ECOG status score of 0 (32/113, 28.3%) or 1 (68/113, 60.2%). Seventy patients had CrCl>60 mL/min, fourteen patients had CrCl 45-60 mL/min, and twenty six had CrCl<45 mL/min. Only five patients had CrCl≦30 mL/min. The average daily dose of POM (4 mg) and the relative dose intensity (0.9) were similar across the three renal groups. Median time to first POM dose reduction by renal group was 49.5 days, 71.0 days, and 32.5 days, respectively; treatment duration was 5.5 months, 5.0 months, and 3.4 month, in patients with CrCl>60 mL/min, CrCl 45-60 mL/min, and CrCl<45 mL/min. Grade ¾ TEAEs occurring in ≧10% of patients are presented in Table 1. Grade ¾ neutropenia was observed in 40% patients with CrCl>60 mL/min, 21% of patients with CrCl 45-65 mL/min, and 54% of patients with CrCl<45 mL/min. Grade ¾ anemia and thrombocytopenia were observed in 19%, 21%, 35%, and 20%, 14%, and 15%, respectively, for patients with CrCl>60 mL/min, CrCl 45-60 mL/min, and CrCl<45 mL/min. Frequently observed non-hematological grade ¾ AEs included pneumonia and fatigue, which was observed in 24%, 21%, 19%, and 14%, 29%, 8% of patients, respectively, for patient with CrCl>60 mL/min, CrCl 45-60 mL/min, and CrCl<45 mL/min.

It was observed that adverse events observed with POM given at 4 mg/day on days 1-21 of each 28-day cycle in combination with LoDEX were generally comparable regardless to baseline renal function.

TABLE 1

| CrCl | | |
|---|---|---|
| >60 mL/min | 45-60 mL/min | <45 mL/min |
| (n = 70) | (n = 14) | (n = 26) |

Grade 3/4 TEAEs occurring in >10% of patients (%)

| | | | |
|---|---|---|---|
| Neutropenia | 40 | 21 | 54 |
| Anemia | 19 | 21 | 35 |
| Thrombocytopenia | 20 | 14 | 15 |
| Leukopenia | 10 | 14 | 4 |
| Pneumonia | 24 | 21 | 19 |
| Urinary tract infection | 6 | 7 | 19 |
| Fatigue | 14 | 29 | 8 |
| Dyspnea | 10 | 7 | 23 |
| Back pain | 10 | 7 | 12 |
| Septic shock | 0 | 14 | 0 |
| Hyperkalemia | 1 | 0 | 12 |

Other Grade 3/4 AEs of interest (%)

| | | | |
|---|---|---|---|
| Febrile neutropenia | 1 | 0 | 4 |
| Pulmonary embolism | 1 | 0 | 4 |
| Deep vein thrombosis | 1 | 0 | 4 |
| Rash | 1 | 0 | 0 |

### 6.2 Phase 1 Clinical Study

A phase 1 multi-center, open-label dose-escalation study was conducted to determine the pharmacokinetics and tolerability of POM in combination with Low-DEX in patients with relapsed/refractory multiple myeloma (RRMM) and renal impairment. The clinical trial followed a 2-stage design. Cohorts A and B of Stage 1 evaluated RRMM patients (≧1 prior therapy) with mild or no renal impairment, kidney function considered normal for their disease (creatinine clearance CrCl≧60 mL/min; n=8 planned) or with severe renal impairment (CrCl<30 mL/min; n=14 planned) not requiring dialysis, respectively. Patients in cohort A were treated with POM at 4 mg and cohort B received POM at 2 or 4 mg on days 1-21 of a 28 day cycle following a standard 3+3 dose escalation design. Both cohorts received DEX at 40 mg (20 mg for patients >75 years of age) on days 1, 8, 15, and 22. Patients were not permitted to enroll in more than one cohort. Treatment was continued until progressive disease or unacceptable toxicity. Stage 2 accesses patients with severe renal impairment requiring dialysis (n=14 planned).

Two patients were enrolled in cohort A and 1 patient in cohort B. The patients in cohort A were ages 65 and 69 years, with 1 and 2 prior therapies, respectively. The patient in cohort B, age 64 years, had 2 prior therapies. CrCl was 68 and 77 mL/min for the two patients in cohort A, respectively, and 18 mL/min for the patient in cohort B. The patient in cohort B completed three cycles without dose-limiting toxicities.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

### 6.3 Pomalidomide+Low-Dose Dexamethasone (POM+LoDEX) Vs. High-Dose Dexamethasone (HiDEX) in Relapsed/Refractory Multiple Myeloma (RRMM): Analysis of Patients with Moderate Renal Impairment (RI)

Patients must have failed lenalidomide (LEN) and bortezomib (BORT) treatment after ≧2 consecutive cycles of each (alone or in combination) and must have been refractory to the last prior LEN and BORT treatment (progressive disease [PD] during the LEN and BORT treatment or within 60 days). Patients with creatinine clearance (CrCl)<45 mL/min were excluded. Randomization was 2:1 to POM 4 mg (Day 1 to Day 21)+DEX 40 mg (20 mg for patients aged >75 y) qw; or DEX 40 mg (20 mg for patients aged >75 y) (Day 1-Day 4, Day 9-Day 12, and Day 17-Day 20) in a 28-D cycle. Treatment continued until PD or unacceptable adverse event (AE). The primary endpoint was progression-free survival (PFS). Secondary endpoints included overall survival (OS) and AEs. This analysis examined patients with or without moderate RI (CrCl<60 vs.≧60 mL/min).

Three hundred two (302) patients received POM+LoDEX; one hundred fifty three (153) patients received HiDEX, of which 31% and 39% had moderate RI, respectively. Patients with moderate RI were more likely to be older (64% vs. 36% aged >65 y) vs. no moderate RI. Median follow-up was 4 mo. As shown in Table 2, median PFS and OS were significantly longer with POM+LoDEX vs. HiDEX, regardless of RI. The most common Gr ¾ AEs (POM+LoDEX vs. HiDEX) for normal renal function: neutropenia (41% vs. 15%), anemia (24% vs. 26%), and infection (23% vs. 23%). Similar AE rates were seen for moderate RI: neutropenia (44% vs. 15%), anemia (33% vs. 34%), and infection (28% vs. 24%). Discontinuation due to AE was 5% vs. 7% (no moderate RI) and 11% vs. 5% (moderate RI).

TABLE 1

| | CrCl | | | | | |
|---|---|---|---|---|---|---|
| | ≧60 mL/min | | | <60 mL/min | | |
| | POM + | | HR | POM + | | HR |
| | LoDEX | HiDEX | (P Value) | LoDEX | HiDEX | (P Value) |
| Median | 3.7 | 1.8 | 0.47 | 3.2 | 1.6 | 0.44 |
| PFS (mo) | | | (<0.001) | | | (<0.001) |
| Median | Not | 9.2 | 0.57 | 10.3 | 4.6 | 0.51 |
| OS (mo) | reached | | (<0.021) | | | (<0.008) |

POM+LoDEX significantly extended PFS and OS vs. HiDEX in patient with moderate RI. Tolerability of POM+LoDEX was acceptable across subgroups, with few discontinuations due to AEs.

What is claimed is:

1. A method of treating, preventing, or managing one or more symptoms of cancer in a subject with renal impairment, comprising administering to the subject a therapeutically effective amount of pomalidomide, wherein the therapeutically effective amount is an amount lower than the amount administered to a patient without renal impairment.

2. The method of claim 1, wherein the cancer is hematological cancer.

3. The method of claim 2, wherein the cancer is multiple myeloma.

4. The method of any of claims 1 to 3, wherein the cancer is drug-resistant.

5. The method of any of claims 1 to 4, wherein the cancer is relapsed or refractory.

6. The method of any of claims 1 to 5, wherein the therapeutically effective amount of pomalidomide is in the range from about 1 mg to about 50 mg per day.

7. The method of claim 6, wherein the therapeutically effective amount of pomalidomide is in the range from about 2 mg to about 25 mg per day.

8. The method of claim 6, wherein the therapeutically effective amount of pomalidomide is in the range from about 2 mg to about 15 mg per day.

9. The method of claim 6, wherein the therapeutically effective amount of pomalidomide is about 2, about 4, or about 6 mg per day.

10. The method of claim 6, wherein the therapeutically effective amount of pomalidomide is about 2 mg per day.

11. The method of claim 6, wherein the therapeutically effective amount of pomalidomide is about 4 mg per day.

12. The method of any of claims 1 to 11, wherein pomalidomide is administered once a day.

13. The method of any of claims 1 to 12, wherein pomalidomide is administered for 21 days in a 28 day treatment cycle.

14. The method of claim 13, wherein pomalidomide is administered daily on days 1 to 21 in the 28 day treatment cycle.

15. The method of any of claims 1 to 14, wherein pomalidomide is administered orally.

16. The method of any of claims 1 to 15, wherein the subject has creatinine clearance of no greater than about 80 mL/min.

17. The method of claim 16, wherein the subject has mild renal impairment.

18. The method of claim 16, wherein the subject has moderate renal impairment.

19. The method of claim 16, wherein the subject has severe renal impairment.

20. The method of any of claims 1 to 19, wherein the subject is a human.

21. The method of any of claims 1 to 20, further comprising administering to the subject a second therapeutic agent.

22. The method of claim 21, wherein the second therapeutic agent is administered in a subtherapeutically effective amount.

23. The method of claim 21 or 22, wherein the second therapeutic agent is dexamethasone.

24. The method of claim 23, wherein dexamethasone is administered in a subtherapeutically effective amount.

25. The method of claim 23 or 24, wherein dexamethasone is administered in the amount ranging from about 10 to about 100 mg per week.

26. The method of claim 25, wherein dexamethasone is administered in the amount ranging from about 10 to about 50 mg per week.

27. The method of claim 25, wherein dexamethasone is administered in the amount of about 20 or about 40 mg per week.

28. The method of claim 25, wherein dexamethasone is administered in the amount of about 20 mg per week.

29. The method of claim 25, wherein dexamethasone is administered in the amount of about 40 mg per week.

30. The method of any of claims 23 to 29, wherein dexamethasone is administered once a day.

31. The method of any of claims 23 to 30, wherein dexamethasone is administered for 4 days in a 28 day treatment cycle.

32. The method of claim 31, wherein dexamethasone is administered daily on days 1, 8, 15, and 22 in the 28 day treatment cycle.

33. The method of any of claims 23 to 30, wherein dexamethasone is administered for 12 days in a 28 day treatment cycle.

34. The method of claim 33, wherein dexamethasone is administered daily on days 1 to 4, 9 to 12, and 17 to 20 in the 28 day treatment cycle.

35. The method of any of claims 23 to 34, wherein dexamethasone is administered orally.

36. The method of any of claims 23 to 35, wherein pomalidomide is administered before dexamethasone.

37. The method of any of claims 23 to 35, wherein pomalidomide is administered concurrently with dexamethasone.

38. The method of any of claims 23 to 35, wherein pomalidomide is administered after dexamethasone.

39. The method of any of claims 21 to 38, further comprising administering to the subject a third therapeutic agent.

40. The method of any of claims 1 to 39, wherein the subject has not been treated with anticancer therapy for the cancer prior to the administration of pomalidomide.

41. The method of any of claims 1 to 39, wherein the subject has been treated with anticancer therapy for the cancer prior to the administration of pomalidomide.

42. The method of claim 41, wherein the subject has been treated with at least two therapies for the cancer prior to the administration of pomalidomide.

43. The method of claim 41, wherein the subject has been treated with lenalidomide or bortezomib for the cancer prior to the administration of pomalidomide.

44. The method of claim 41, wherein the subject has been treated with lenalidomide and bortezomib for the cancer prior to the administration of pomalidomide.

45. A method of inhibiting the growth of a cell, comprising contacting the cell with pomalidomide and dexamethasone.

46. The method of claim 45, wherein pomalidomide is contacted with the cell before the dexamethasone.

47. The method of claim 45, wherein pomalidomide is contacted with the cell concurrently with dexamethasone.

48. The method of claim 45, wherein pomalidomide is contacted with the cell after dexamethasone.

49. The method of any of claims 45 to 48, wherein the cell is a cancerous cell.

50. The method of claim 49, wherein the cancerous cell is a cell of bladder cancer, breast cancer, cervical cancer, colon cancer, endometrial cancer, gastric cancer, glioma, head and neck cancer, liver cancer, non-small cell lung cancer, ovarian cancer, pancreatic cancer, or prostate cancer.
